(19) United States
(12) Patent Application Publication
(10) Pub. No.: US 2002/0193343 A1
(43) Pub. Date: Dec. 19, 2002

(54) Controlled enzymatic degradation of guar galactomannan solutions using enzymatic inhibition

(76) Inventors: Saad A. Khan, Cary, NC (US); Robert M. Kelly, Cary, NC (US); Robert K. Prud'homme, Lawrenceville, NJ (US); Matthew D. Burke, Carrboro, NC (US); Yu Cheng, Princeton, NJ (US); Swapnil Chhabra, Raleigh, NC (US)

Correspondence Address: MYERS BIGEL SIBLEY & SAJOVEC, PO BOX 37428, RALEIGH, NC, 27627, US

(21) Appl. No.: 09/951,099

(22) Filed: Sep. 11, 2001

Related U.S. Application Data
(60) Provisional application No. 60/235,762, filed on Sep. 27, 2000.

(51) Int. Cl. A61K 31/715; A01N 43/04; C12N 9/00

(52) U.S. Cl. 514/54; 435/183

(57) ABSTRACT

A method of controlling enzymatic degradation of guar galactomannan with β-mannanase by selectively inhibiting β-mannanase, in a pH-dependent or ionic strength-dependent manner, with an aminoglycol such as TRIS or an charged polymer agent is described.

### RELATED APPLICATIONS

This application claims the benefit of provisional application Serial No. 60/235,762, filed Sep. 27, 2000, the disclosure of which is incorporated by referenced herein in its entirety.

This invention was made with Government support under Grant No. BES -9707160 (North Carolina State University) and Grant No. BES 97-11781 (Princeton University) from the National Science Foundation. The Government has certain rights to this invention.

### FIELD OF THE INVENTION

This invention concerns controlling the activity of thermostable enzyme breakers for the hydrolysis of polysaccharides in hydraulic fracturing fluids.

### BACKGROUND OF THE INVENTION

When the pressure of oil or gas in a reservoir declines as oil or gas is taken from that reservoir, production from a well in that reservoir declines. Hence, the economic viability of the well declines until it is no longer profitable to operate (even though it continues to produce gas or oil). Production can be increased from such wells through oil well stimulation. In addition, where forming a bore hole into a reservoir is very expensive, such as in offshore drilling, it is desirable to stimulate production from a single well.

Oil well stimulation typically involves injecting a fracturing fluid into the well bore at extremely high pressures to create fractures in the rock formation surrounding the bore. The fractures radiate outwardly from the well bore, typically from 100 to 1000 meters, and extend the surface area from which oil or gas drains into the well. The fracturing fluid typically carries a propping agent, or “proppant”, such as sand, so that the fractures are propped open when the pressure on the fracturing fluid is released, and the fracture closes around the propping agent. This leaves a zone of high permeability (the propping agent trapped and compacted in the fracture in the subterranean formation.

The fracturing fluid typically contains a water-soluble polymer, such a guar gum or a derivative thereof, which provides appropriate flow characteristics to the fluid and suspends the proppant particles therein. When pressure on the fracturing fluid is released and the fracture closes around the propping agent, water is forced therefrom and the water-soluble polymer forms a compacted cake. This compacted cake can prevent oil or gas flow if not removed. To solve this problem, “breakers” are included in the fracturing fluid.

Currently, breakers are either enzymatic breakers or oxidative breakers. The enzyme breakers are preferable, because (a) they are true “catalysts”, (b) they are relatively high in molecular weight and do not leak off into the surrounding formation, and (c) they are less susceptible to dramatic changes in activity by trace contaminants. Oxidative breakers, on the other hand, are low in molecular weight and leak off into the formation, and they are active only over a very narrow temperature range.

Effective use of enzymes to modify guar requires that the onset of enzymatic hydrolysis be controlled. Currently, the enzyme and guar solutions are mixed together before injection into the oil well. This could allow premature enzyme degradation of guar to occur thereby decreasing the guar gel's ability to fracture the subterranean formations. One approach to solving this problem is described in U.S. Pat. No. 5,869,435 to Kelly et al., which provides an enzyme breaker that is active primarily at the higher temperatures found in deep wells. Nevertheless, there remains a need for additional ways to provide selectively active enzyme breakers.

### SUMMARY OF THE INVENTION

A first aspect of the present invention is a method of fracturing a subterranean formation which surrounds a well bore, comprising the steps of:

(a) providing a fracturing fluid comprising (i) an aqueous liquid; (ii) a polysaccharide soluble or dispersible in the aqueous liquid in an amount sufficient to increase the viscosity of the aqueous liquid; (iii) an enzyme breaker which degrades the polysaccharide; and (iv) a compound according to Formula I in an amount sufficient to reduce the polysaccharide-degrading activity of the enzyme breaker;

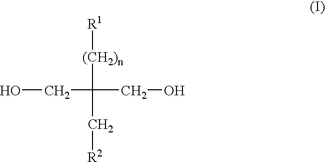

wherein:

R¹ is selected from the group consisting of: —F, —NR³R⁴ wherein R³ and R⁴ are each independently selected from the group consisting of H, loweralkyl,

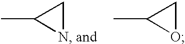

R² is selected from the group consisting of —H and —OH; and

n is 0 to 3;

then;

(b) injecting the fracturing fluid into the well bore at a pressure sufficient to form fractures in the subterranean formation which surrounds the well bore; then

(c) reducing the pH of the fracturing fluid by an amount sufficient to increase the polysaccharide-degrading activity of the enzyme; and then

(d) releasing the pressure from the fracturing fluid.

A second aspect of the present invention is a fracturing fluid useful for fracturing a subterranean formation which surrounds a well bore, comprising:

(i) an aqueous liquid;

(ii) a polysaccharide soluble or dispersible in the aqueous liquid in an amount sufficient to increase the viscosity of the aqueous liquid;

(iii) an enzyme breaker which degrades the polysaccharide; and

(iv) a compound according to Formula I as described above in an amount sufficient to reduce the polysaccharide-degrading activity of the enzyme breaker.

A third aspect of the present invention is a method of fracturing a subterranean formation which surrounds a well bore, comprising the steps of:

(a) providing a fracturing fluid comprising (i) an aqueous liquid; (ii) a polysaccharide soluble or dispersible in the aqueous liquid in an amount sufficient to increase the viscosity of the aqueous liquid; (iii) an enzyme breaker which degrades the polysaccharide, and (iv) a polymeric additive which has a positive charge to decrease the activity of the enzyme breaker under conditions where the enzyme has a negative charge, subject to the proviso that the polymeric additive and the polysaccharide may be different or the same;

(b) injecting the fracturing fluid into the well bore at a pressure sufficient to form fractures in the subterranean formation which surrounds the well bore; then

(c) reducing the pH and/or increasing the ionic strength of the fracturing fluid by an amount sufficient to increase the polysaccharide-degrading activity of the enzyme; and then

(d) releasing the pressure from the fracturing fluid. The polymeric additive is positively charged by virtue of carrying cationic moities such as amines, quaternary amines and sulfonium ions. Note that copolymers of cationic and nonionic monomers may be used, for example, guar with partial cationic substitution or copolymers of acrylamide and cationic monomers.

A fourth aspect of the present invention is a method of fracturing a subterranean formation which surrounds a well bore, comprising the steps of:

(a) providing a fracturing fluid comprising (i) an aqueous liquid; (ii) a polysaccharide soluble or dispersible in the aqueous liquid in an amount sufficient to increase the viscosity of the aqueous liquid; (iii) an enzyme breaker which degrades the polysaccharide, and (iv) a polymeric additive which has a negative charge to decrease the activity of the enzyme breaker under conditions where the enzyme has a positive charge, subject to the proviso that the polymeric additive and the polysaccharide may be different or the same;

(b) injecting the fracturing fluid into the well bore at a pressure sufficient to form fractures in the subterranean formation which surrounds the well bore; then

(c) reducing the pH and/or increasing the ionic strength of the fracturing fluid by an amount sufficient to increase the polysaccharide-degrading activity of the enzyme; and then

(d) releasing the pressure from the fracturing fluid.

The polymeric additive is negatively charged by virtue of carrying anionic moities such as carboxylic, sulfate, sulfonate, phosphate and phosphonate ions. Note that copolymers of anionic and nonionic monomers may be used.

A fifth aspect of the present invention is a fracturing fluid useful for fracturing a subterranean formation which surrounds a well bore, comprising:

(i) an aqueous liquid;

(ii) a polysaccharide soluble or dispersible in the aqueous liquid in an amount sufficient to increase the viscosity of the aqueous liquid;

(iii) an enzyme breaker which degrades the polysaccharide;

(iv) a polymeric additive which has an opposite charge to that of the enzyme to decrease the activity of the enzyme breaker, subject to the proviso that the polymeric additive and the polysaccharide may be different or the same.

The polymeric additive is charged by virtue of carrying an ionic group such as a sulfate, sulfonate, phosphate, phosphonate, carboxylate, amine, quaternary amine or sulfonium ion.

The foregoing and other objects and aspects of the present invention are explained in greater detail in the drawings herein and the specification set forth below.

### BRIEF DESCRIPTION OF THE DRAWINGS

FIG. 1 shows the effect of pH on the enzymatic hydrolysis (by Aspergillus niger βmannanase, 8.3×10⁻⁴ U/ml) of 7 mg/ml guar solutions as determined by the viscosity reduction factor (●, left y-axis). The pH profile of the enzyme activity (◯, right y-axis), as determined by a colorimetric technique (P. Ademark et al., Journal of Biotechnology, 63 (1998) 199-210), shows a similar trend and validates viscosity reduction as an effective technique to measure enzyme activity.

FIG. 2 shows viscosity versus shear rate behavior of 7 mg/ml guar solutions containing various amounts of TRIS, degraded by 8.3×10⁴ U/ml β-mannanase for 1 hour at pH 9. The symbol (●) represents the initial (undegraded) viscosity; (◯) the degraded guar solution which contained 0 mM TRIS; (□), 1 mM TRIS; (□), 5 mM TRIS; (♦), 10 mM TRIS; (Δ), 25 mM TRIS; and (×), 50 mM TRIS. Due to overlap of the data for the initial viscosity, 10 mM, 25 mM and 50 mM TRIS samples, some data points are omitted for clarity.

FIG. 3 shows the effect of TRIS on the viscosity profile of 7 mg/ml guar solutions degraded by 8.3×10⁻⁴ U/ml β-mannanase, for 5 hours at pH 9. The symbol (●) represents the initial viscosity; (◯) the degraded guar solution which contained 0 mM TRIS; (▪), 1 mM TRIS; (□), 5 mM TRIS; (♦), 10 mM TRIS; (Δ), 25 mM TRIS; and (×), 50 mM TRIS. Due to overlap of the data for the initial viscosity, 25 mM and 50 mM TRIS samples, some data points are omitted for clarity.

FIG. 4 shows the viscosity profile of 7 mg/ml guar solutions containing various amounts of TRIS and degraded by 8.3×10⁻⁴ U/ml β-mannanase for 1 hour at pH 4. The symbol (●) represents the initial viscosity; (◯) the degraded guar solution which contained 0 mM TRIS; (♦), 10 mM TRIS; (Δ), 25 mM TRIS; and (×), 50 mM TRIS.

FIG. 5 shows the pH activated enzyme degradation of a 7 mg/ml guar solution by Aspergillus niger β-mannanase (8.3×10⁻⁴ U/ml). The pH of the solution was maintained at 9 for the first 5 hours; subsequently, the enzyme was activated by adjusting the pH to 4. Viscosity shear rate behavior are shown for various times during the process. Zero shear viscosity is shown as a function of time in the inset. (●, viscosity at 0 hr; ◯, 1 hr; ▪, 3 hr; □, 5 hr; ♦, 6 hr; Δ, 7 hr; ×, 9 hr; and ∇, 11 hr) Due to the overlap of the data for the 0 hr, 1 hr, 3 hr and 5 hr samples, some data points were omitted for clarity.

FIG. 6 shows the comparison of the viscosity behavior of a 7 mg/ml guar solution (●) to the viscosity profiles of guar solutions degraded with 8.3×10⁻⁴ U/ml β-mannanase for 1 hour at pH 9, and containing 0 mM TRIS (◯), 25 mM of TRIS (Δ), 25 mM of Tris (hydroxymethyl)nitromethane (□)and 25 mM of N-tris (hydroxymethyl)methylglycine (▪). Some data points have been removed for the sake of clarity.

FIG. 7 shows the viscosity profile of guar solutions containing varying amounts of TRIS, degraded by 8.3×10⁻⁴ U/ml β-mannanase for 1 hour at pH 7. The symbol (●) represents the initial viscosity; (◯) the degraded guar solution which contained 0 mM TRIS; (▪), 1 mM TRIS; (□), 5 mM TRIS; (♦), 10 mM TRIS; and (Δ), 25 mM TRIS.

FIG. 8 shows the pH-dependent inhibition of β-mannanase by TRIS plotted in terms of relative activity. The relative activity was obtained from viscosity data and normalized for pH, as described in the experimental section.

FIG. 9 shows the proposed representation of the interaction between TRIS and the acid/base catalytic and histidine residues in the active site of the enzyme. The protonation state of the structures are shown for pH 4, 7 and 9.

FIG. 10 shows the viscosity of 0.5 wt % guar solution as a function of shear rate plotted at different periods during enzymatic degradation. The reaction was run at ambient temperature and a pH of 7. The concentration of β-mannanase is 0.0002 units/ml polymer solution.

FIG. 11 shows the viscosity of 0.5 wt % cationic guar solution versus shear rate plotted at different periods during enzymatic degradation. The reaction was run at ambient temperature and a pH of 7. The concentration of β-mannanase is 0.0002 units/ml polymer solution.

FIG. 12 shows the viscosity of 0.5wt % cationic guar solution as a function of shear rate plotted at different periods during enzymatic degradation. The reaction was run at ambient temperature and a pH of 3.5. The concentration of ,β-mannanase is 0.0002 units/ml polymer solution.

FIG. 13 shows the normalized viscosity of 0.5 wt % guar solution changes with reaction time at pH=7, 3.4 and cationic guar solution at pH=3.5. The concentration of β-mannanase is 0.0002 units/ml polymer solution.

FIG. 14 shows the normalized viscosity of 0.5 wt % cationic guar solution changes with reaction time at different solution ionic strengths. All reactions were run at ambient temperature and pH of 7. The concentration of β-mannanase is 0.0002 units/ml polymer solution.

FIG. 15 shows the effect of salt on the enzyme activity of degrading guar: normalized viscosity of 0.5 wt % guar solution as a function of reaction time at different solution ionic strengths. All reactions were run at ambient temperature and pH of 7. The concentration of β-mannanase is 0.0002 units/ml polymer solution.

### DETAILED DESCRIPTION OF PREFERRED EMBODIMENTS

Fracturing fluids used to carry out the present invention are, in general, prepared from an aqueous base fluid such as water, brine, aqueous foams or water-alcohol mixtures. Any suitable mixing apparatus may be used to provide the fracturing fluid. The fracturing fluid includes a polysaccharide as a gelling agent, as discussed below, and typically includes other ingredients such as proppant particles and crosslinking agents to crosslink the polysaccharide gelling agent, also discussed below.

Polysaccharides soluble or dispersible in an aqueous liquid include industrial gums such as those generally classified as exudate gums, seaweed gums, seed gums, microbial polysaccharides, and hemicelluloses (cell wall polysaccharides found in land plants) other than cellulose and pectins. Examples include xylan, mannan, galactan, L-arabino-xylans, L-arabino-D-glucurono-D-xylans, D-gluco-D-mannans, D-Galacto-D-mannans, arabino-D-galactans, algins such as sodium alginate, carrageenin, fucordan, laminarin, agar, gum arabic, gum ghatti, karaya gum, tamarind gum, tragacanth gum, locust bean gum, cellulose derivative such as hydroxyethylcellulose or hydroxypropylcellulose, and the like. Particularly preferred are the hydratable polysaccharides having galactose and/or mannose monosaccharide components, examples of which include the galactomannan gums, guar gum (including derivatized guar gums such as carboxymethyl guar, hydroxypropyl guar, and carboxymethyl hydroxypropyl guar.

When the polysaccharide is derivatized or substituted with an ionic group (e.g., ionic groups are covalently bound to the polysaccharide), any suitable ionic group may be employed. Particularly preferred are cationic groups, such as quaternary amines or sulfonium ions. Examples are given in U.S. Pat. No. 5,045,190 to Carbonell et al. at column 7, lines 16-35, the disclosure of which is incorporated herein by reference. The extensive literature on ion exchange chromatography may be referred to by those skilled in the art for numerous examples of substrates derivatized with ionic groups.

The amount of polysaccharide included in the fracturing fluid is not particularly critical, so long as the viscosity of the fluid is sufficiently high to keep the proppant particles suspended therein during the injecting step. Thus, depending upon the application, the polysaccharide is included in the fracturing fluid in an amount of from about 10 to 150 pounds of polysaccharide per 1000 gallons of aqueous liquid, and more preferably in an amount of from about 20 to 100 pounds of polysaccharide per 1000 gallons of aqueous solution (about 2.4 to 12 kg/m³).

Control of Enzyme Activity

Enzymatic attack on the polysaccharide chain can be controlled (arrested) by addition of a complexing agent which may be (1) a low molecular weight solute (such as amino glycols or other substituted glycols), or (2) a higher molecular weight oppositely charged polymer (such as cationic guar which interacts with the anionic enzyme). By “arrested” or “reduced activity” is meant that the enzyme is inactive or essentially inactive; some minor enzyme activity is permissible as long as the viscosity of the fracturing fluid does not decrease by 10 or 20 percent or more prior to the step of reducing the pH and/or increasing the ionic strength of the fracturing fluid (e.g., a time of 1, 2, or 4 or 5 hours).

Amino Glycol Control Agents

Aminoglycols or other substituted glycols that may be used to carry out the invention are generally represented by Formula I:

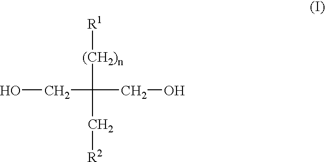

wherein:

R¹ is selected from the group consisting of: —F, —NR³R⁴ wherein R³ and R⁴ are each independently selected from the group consisting of H, loweralkyl,

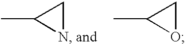

R² is selected from the group consisting of —H and —OH; and

n is 0 to 3 (preferably 1).

Preferably, R¹ is —NH₂ and preferably R² is —OH. A particularly preferred compound of Formula 1 is 2-amino-2-hydroxymethyl-1,3-propanediol, or “TRIS”.

“Loweralkyl” as used herein preferably means C1-C4 alkyl, such as methyl, ethyl, propyl, isopropyl, butyl, tert-butyl, etc.

Compounds as described above can be prepared in any suitable manner, such as described in U.S. Pat. No. 2,174,242, the disclosure of which is incorporated herein by reference.

The ratio of the compound of Formula I to Enzyme in the solution is preferably 0, 1 or 5 to about 500, 600 or 1000. For example, but for a range of values for a 1 liter solution 2.0×10⁻² milligrams of enzyme and Tris between 0 or 1 to 12 grams may be used.

Aminoglycols are reversible inhibitors of the enzymes. For example, at a concentration of 25mM and a pH of 9, TRIS (2-amino-2-hydroxymethyl-1,3-propanediol) inhibits enzyme degradation for an extended period of time, with an enzyme concentration of 8.3×10⁻⁴ U/ml of guar solution. When the pH is shifted to a pH of 4, enzyme degradation is restored and the guar molecules are degraded quickly.

Oppositely Charged Polymer Control Agents

The complexing can also be conducted using an oppositely charged high molecular weight (HMW) agent ( 3,000<Mw<10⁸ ) . The HMW agent, typically a polymer, and the enzyme must be of opposite charge in the “arrested” state, and will initiate degradation either by shifting pH to alter the charge on either the polymer or enzyme, or by adding salts (of any anion or cation type) to free complexed enzyme to initiate degradation. The complexing can be cationically charged polymer and anionic protein, and vice versa. The opposite charged polymer may be used as the viscosifying polymer or it may be a different or separate polymer added to complex the enzyme. Examples of oppositely charged HMW agents would include sulfated, sulfonated phosphated, phosphonated, carboxylated, or amine, quaternary amine or sulfonium ion containing synthetic or natural polymers.

Other Fracturing Fluid Ingredients

Any crosslinking agent may be used to carry out the present invention. Examples include metal ions including aluminum, antimony, zirconium and titanium containing compounds including organotitantates (see, e.g., U.S. Pat. No. 4,514,309). Borate crosslinking agents or borate ion donating materials, are currently preferred. Examples of these include the alkali metal and alkaline earth metal borates and boric acid, such as sodium borate decahydrate. The crosslinking agent is typically included in an amount in the range of from about 0.0245 to 0.18% by weight of the aqueous fluid or more.

Proppant particles or propping agents are typically added to the base fluid prior to the addition of the crosslinking agent. Propping agents include, for example, quart sand grains, glass and ceramic beads, walnut shell fragments, aluminum pellets, nylon pellets, and the like. The propping agents are typically included in an amount of from 1 to 8 or even 18 pounds per gallon of fracturing fluid composition. Particle size of the proppant particles is typically in the range of about 200 to about 2 mesh on the U.S. Sieve Series scale. The base fluid may also contain other conventional fracturing fluid additives, such as buffers, surfactants, antioxidants, corrosion inhibitors, bactericides, etc.

Enzyme breaker compositions useful for carrying out the present invention may be provided in any suitable physical form, such as concentrated or dilute aqueous solutions, lyophylized powders, etc. The compositions contain an enzyme effective for degrading the particular crosslinking polysaccharide employed as the gelling agent. Enzyme breakers are typically β-mannanases or β-glucosidases, which may be thermophilic or mesophilic and may be obtained from any suitable organism. Examples include, but are not limited to, those described in U.S. Pat. No. 5,896,435 to Kelly et al., the disclosure of which is incorporated by reference herein in its entirety. In general, the amount of enzyme will be between about 0.0005 or 0.001-0.004 or 0.01 percent by weight based on the total weight of aqueous solution Such enzymes are available from Megazyme International Ireland Ltd., Novo Nordisk of Norway, Diversa Corporation, San Diego, Calif. as well as Sigma and many other companies.

The present invention may be carried out on subterranean formations which surround any type of well bore, including both oil and gas well bores, with the fracturing fluid being provided and injected and pressure released, etc., all in accordance with procedures well known to those skilled in the art.

Adjusting pH

When pH is adjusted to activate the enzyme, in general, prior to acidification, the pH range of the fracturing fluid will be about 7, 8 or 9 to 10 or more, and will be from about 2 or 3 to 6 after acidification.

In the well bore, it would be useful to initiate the enzyme reaction at a particular time, however, adjusting the pH after the hydraulic fracturing fluid is in the well bore can be difficult to almost impossible. After injection, the fracturing fluid may be acidified by any suitable means. An acidic solution can be injected into the well bore after injection of the fracturing fluid. Pressure-sensitive capsules containing an acid can be included in the fracturing fluid, with the capsules selected to break and release their contents at the pressure encountered by the fracturing fluid upon injection. Temperature-sensitive capsules containing an acid can be included in the fracturing fluid, with the capsules selected to break and release their contents at the temperatures encountered by the fracturing fluid after injection. Water-soluble capsules containing an acid can be added to the fracturing fluid shortly before injection, or at the time of injection, so that the capsules dissolve after injection. An enzyme such as a esterase/lipases could be included in the fracturing fluid along with its appropriate substrate (esters/lipids). As the esterase/lipase reaction proceeds, the pH will be reduced. In fact, the pH change of these type of reactions are often used to quantify enzyme activity for these systems. A general schematic of the reaction is shown below.

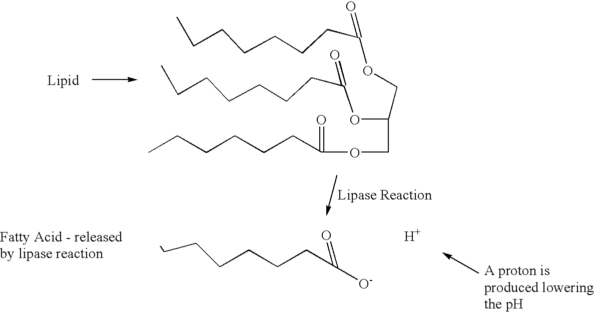

Note that TRIS is temperature sensitive and can produce a drop of about−0.028 pH units for each degree increase in the temperature. Thus, a temperature increase from 25 degrees C. to 60 degrees C. (the temperature optimum of the enzyme) will drop the pH by 0.98. While this does not shift the pH enough to activate the enzyme, it does assist in dropping the pH for well bores with temperatures higher than ambient conditions (Bates and Bower. Analyt. Chem, 28, 1322 (1956)).

The present invention is explained in greater detail in the following non-limiting Examples, where “U” means unit of enzyme activity, “ml” means milliliter, “mg” means milligram, “rpm” means revolutions per minute, “min” means minute, “mM” means milliMolar, and temperatures are given in degrees Centigrade unless otherwise indicated, “μ” means microliter, “mm” means millimeter, “η” means viscosity, “Δη” means viscosity difference, “g” means gram, “ppm” means parts per million, “HCl” means hydrochloric acid, “M” means Molar, “M_{W}” means molecular weight, “c[η]” means specific viscosity against degree of space occupancy, “dL” means deciliter, “ηsp” means specific viscosity, “Pa·s” means pascals seconds.

### EXAMPLE 1

### pH-Dependent Inhibition of β-Mannanase by TRIS

Experimental: Tris(hydroxymethyl)aminomethane, Tris(hydroxymethyl)aminomethane hydrochloride, glycine, sodium acetate, sodium phosphate, 2-(4-morphoino)-ethane sulfonic acid (MES) and sodium azide were purchased from Sigma. N-Tris(hydroxymethyl)methylglycine, Tris(hydroxymethyl)nitromethane and guar gum were purchased from Aldrich. Aspergillus niger β-mannanase (Megazyme, Ireland, Lot 50401, 41 U/mg, 297 U/ml) was used without further purification.

Purification of Guar: Guar was sprinkled slowly into the vortex of water to a concentration of 7 mg/ml. This solution was vigorously mixed for 1 hour followed by low shear mixing for 24 hours. The solution was then centrifuged at 7000 rpm for 30 min. The supernatant was collected and 2 volumes of ethanol were added. The precipitate was collected and lyophilized for 48 hours. The purified guar was then redissolved in de-ionized water to a concentration of 7 mg/ml. Sodium azide at 0.2 mg/ml was added as a biocide.

Enzyme Degradation Assay: Enzyme degradation was conducted with 7 mg/ml purified guar solutions containing 0.2 mg/ml sodium azide and 20 mM of the appropriate buffer. Guar solutions (19 ml) and enzyme stock solution were separately pre-incubated at 25° C. for 15 minutes, followed by the addition of 78.9 μl of enzyme stock solution to 19 ml of guar solution. The final enzyme concentration was 8.3×10⁻⁴ U/ml. The mixture was shaken at an agitation speed of 2 at 25° C. for 1 hour in a New Brunswick gyrotory water bath shaker (Model G76). The enzyme was denatured after 1 hour by placing the enzyme/guar solution in a 90° C. water bath for 10 minutes. Enzyme hydrolysis was conducted at different pH's using various buffers: sodium acetate was used for pH 4 and 5, MES was used for pH 6, sodium phosphate was used for pH 7 and pH 8 and glycine was used for pH 9. If inhibitor, inhibitor variant or NaCl was used, the appropriate amount was added to the guar solution and the pH was readjusted to the appropriate value. To check for irreversible inhibition, solutions were prepared by adding TRIS to a 0.2 U/ml enzyme solution in TRIS to enzyme molar ratio of 1:1 and 5:1. After incubating each of these solutions at 25° C. for 30 minutes, 78.9 μl of this solution was added to 19 ml of guar solution and the enzymatic degradation of guar was analyzed following known techniques.

Rheology: Steady shear experiments were conducted using a Rheometrics Dynamic Stress Rheometer (DSR II) and a couette fixture with a cup diameter of 31.9 mm, a bob diameter of 29.5 mm and a bob length of 44.25 mm. All experiments were performed at 25° C. and all reported data were reproducible within 10%. Steady shear experiments were performed to determine viscosity, η, as a function of shear rate. A viscosity reduction factor was determined by dividing the zero shear (Newtonian) viscosity of the undegraded guar solution by the zero shear viscosity of the degraded guar solution, which had been treated with the enzyme for 1 hour. To analyze a portion of the data, a normalized relative activity was defined using the viscosity data. At a particular pH, the viscosity difference (Δη) between the initial zero shear viscosity and the zero shear viscosity after 1 hour of degradation was used as the reference Δη. At the same pH, the difference between the initial zero shear viscosity and zero shear viscosity after 1 hour of degradation with TRIS was then divided by the reference Δη and multiplied by 100 to calculate the normalized relative activity. In summary,

$$relative activity = \frac{{η}_{o} ( noEnzyme , noTRIS ) - {η}_{o} ( w / Enzyme , w / TRIS )}{{η}_{o} ( noEnzyme , noTRIS ) - {η}_{o} ( w / Enzyme , noTRIS )} \times 100$$

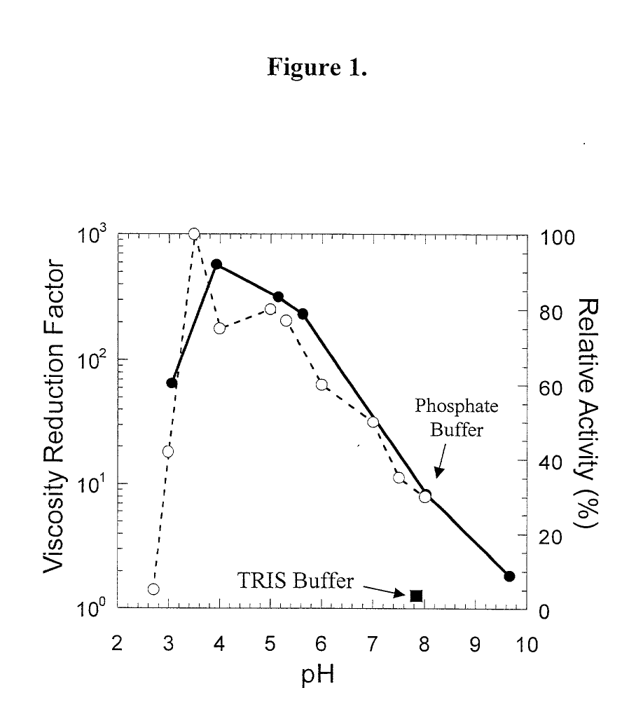

Results: The effect of the solution pH on the enzymatic degradation of guar solutions is shown in FIG. 1 in terms of the viscosity reduction factor (ratio of undegraded solution viscosity to degraded solution viscosity). The viscosity reduction factor was determined at each pH following incubation of the guar with Aspergillus niger β-mannanase for one hour. Various buffers were used to control the pH. β-mannanase was found to be most effective at pH ˜4 with a viscosity reduction factor of ˜600. Also, shown in FIG. 1 is the effect of pH on the relative enzyme activity obtained using a colorimetric assay (Ademark et al., Journal of Biotechnology, 63 (1998) 199-210). The similarity between the two experimental techniques indicates that the viscosity reduction factor is an effective measure of enzyme activity.

FIG. 1 demonstrates differences in enzyme activity, obtained at pH 8, using two different buffers: sodium phosphate and tris(hydroxymethyl)aminomethane hydrochloride (TRIS). When sodium phosphate was used as the buffer, degradation was detected by both the viscosity reduction factor and the colorimetric assay. However, when TRIS was used, a negligible reduction in viscosity was observed, suggesting that TRIS may be an inhibitor of Aspergillus niger β-mannanase thus preventing the enzymatic degradation of guar.

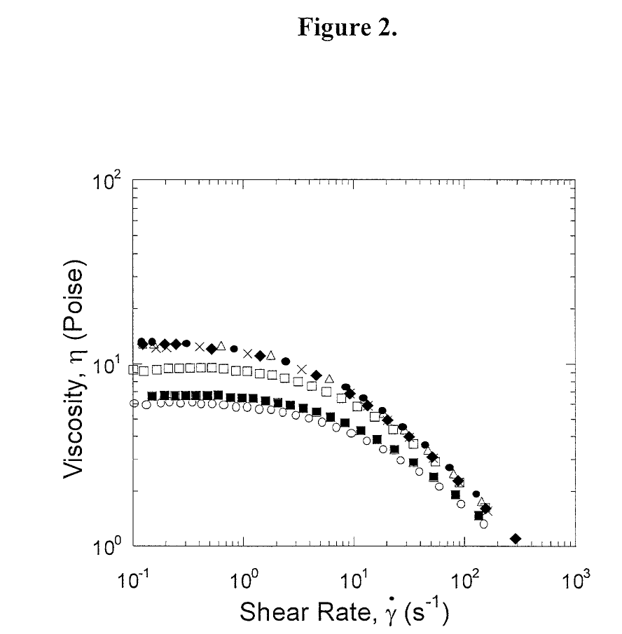

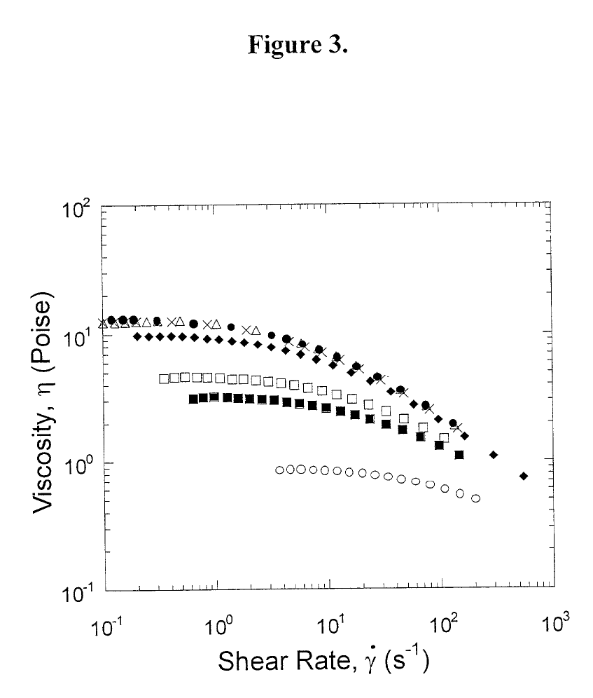

To probe the potential inhibition of β-mannanase by TRIS, viscosity profiles of guar solutions at pH 9 were examined following one hour of enzymatic degradation in the presence of TRIS. FIG. 2, shows that the addition of 1 mM TRIS produced only minimal inhibition of β-mannanase. However, increasing the amount of TRIS to 5 mM inhibited the degradation considerably and samples with 10 mM and higher amounts of TRIS displayed no viscosity reduction. To test how these trends progressed with time, the viscosity of guar solutions were measured after 5 hours of β-mannanase treatment (FIG. 3). It was found that even 1 mM of TRIS impeded viscosity reduction considerably. Samples with 25 mM and higher amounts of TRIS showed no viscosity reduction and samples with 10 mM showed minimal viscosity reduction. Thus, at high pH (e.g. 9), the extent of enzyme degradation could be clearly controlled by the amount of TRIS added to the guar solution.

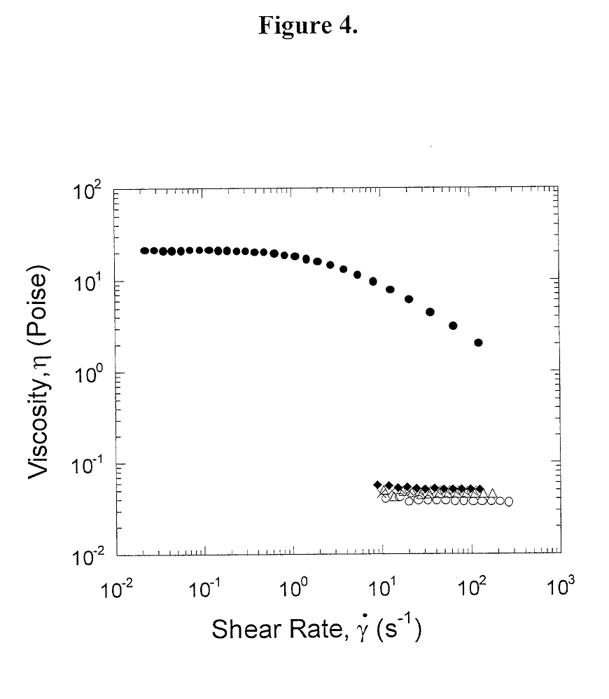

The effect of TRIS on enzyme degradation was examined at pH 4, the optimum pH of the enzyme (Ademark et al., Journal of Biotechnology, 63 (1998) 199-210; McCleary, β-D-Mannanase, in: W. A. Wood, S. T. Kellogg, (Ed.), Methods in Enzymology, Academic Press, Inc., San Diego (1988) p. 596-611). FIG. 4 shows the viscosity profile after 1 hour of enzymatic hydrolysis of guar solutions containing 0-25 mM TRIS. At pH 4, all samples showed significant reduction in viscosity. No difference in viscosity was found between samples containing TRIS and those without TRIS. This indicated that the TRIS did not impede enzymatic hydrolysis by ,β-mannanase at pH 4. One possible explanation for the pH-dependent inhibition could be that the enzyme was more sensitive to ionic strength at pH 9 rather than pH 4 resulting in the pH-dependent inhibition. As TRIS-HCl was used, the additional chloride ions increased the ionic strength. To determine if ionic strength sensitivity was the cause of the inhibition, the base form of TRIS (without any salts) was purchased and tested as an inhibitor of β-mannanase. The results were exactly the same as that of TRIS in its hydrochloride form. Next, NaCl was added to the solutions at pH 4 and pH 9 in the same quantities that were present when using TRIS in its hydrochloride form. The additional salt did not alter the viscosity reduction by the enzyme (data not shown). The pH-dependent inhibition of the enzymatic degradation of TRIS allowed development of a pH-activated enzyme degradation. This type of control applicable in industrial applications where mixing of the enzyme and guar solutions is preferred, yet degradation is only needed after the solution's high viscosity has served its purpose (Cipolla et al. SPE Production and Facilities, 11 (1996) 216-222; J. A. Menjivar, Use of Gelation Theory to Characterize Metal Cross-Linked Polymer Gels, in: J. E. Glass, (Ed.), Water-Soluble Polymers, American Chemical Society, Washington, D.C., 1986, p. 209-226).

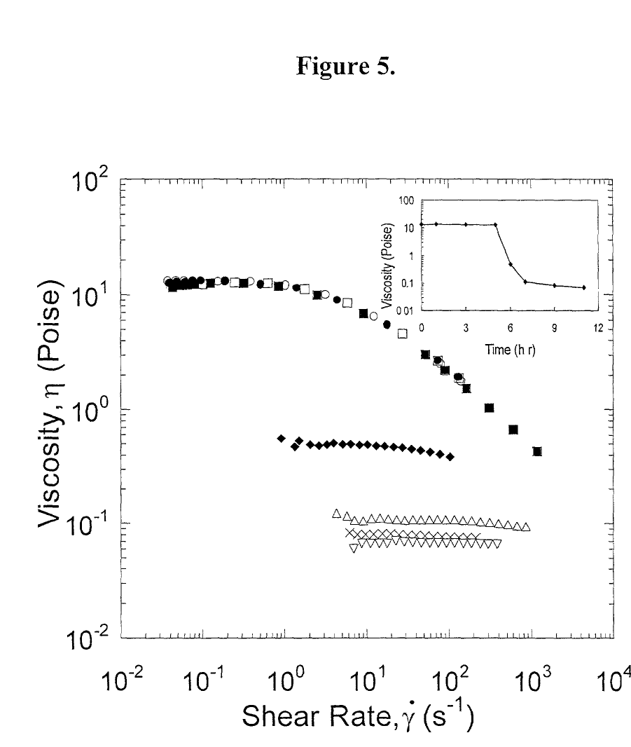

pH Activated Enzyme Degradation: An effective pH-activated enzyme degradation is one in which no degradation takes place until the pH is changed. Without the presence of an inhibitor, a simple pH change from 9 to 4 altered the relative activity of the enzyme from 20% to 100%, based on the pH profile of the enzyme. Though there was already a pH-activated enzyme degradation process because of the large change in enzyme activity, an enzyme with 20% relative activity produces significant reduction in guar viscosity (FIG. 3) even though it was not acting at its optimum level. The rapid, initial rate of viscosity reduction was due to the unique “hyper-entanglements” found in guar solutions that produces a stronger dependence of viscosity on molecular weight than that found in other biopolymers (Tayal et al., Enzymatic Modification of Guar Solutions: Viscosity—Molecular Weight Relationships, In: Amjad, (Ed.), Water Soluble Polymers, Plenum Press 1998, p. 41-49). Using TRIS inhibition in combination with a pH change, allowed a pH-activated enzyme degradation to be achieved. A 25 mM TRIS guar solution at pH 9 was prepared and enzyme was added. A pH of 9 was maintained for 5 hours after which the pH was adjusted to 4. FIG. 5 displays the viscosity profile at various time intervals of the degradation process. It shows that TRIS was able to effectively prevent enzyme degradation of the guar during the first 5 hours and the enzyme could be activated by reducing the pH to 4. After the pH was adjusted to 4, the viscosity reduction proceeded quickly during the first hour followed by a slower decrease at longer times. This shows that TRIS can effectively be used for a pH-activated enzyme degradation.

The fact that enzymatic degradation could be triggered after 5 hours following a pH shift indicated that TRIS was a reversible inhibitor. This was further verified by mixing TRIS directly with the enzyme solution for 30 minutes prior to adding the enzyme to the guar solutions. Two different molar ratios of TRIS to enzyme were used, 1:1 and 5:1, however, neither of these solutions showed any inhibition (data not shown). If TRIS were an irreversible inhibitor, the TRIS would have been permanently bound to the enzyme and viscosity reduction would not have occurred.

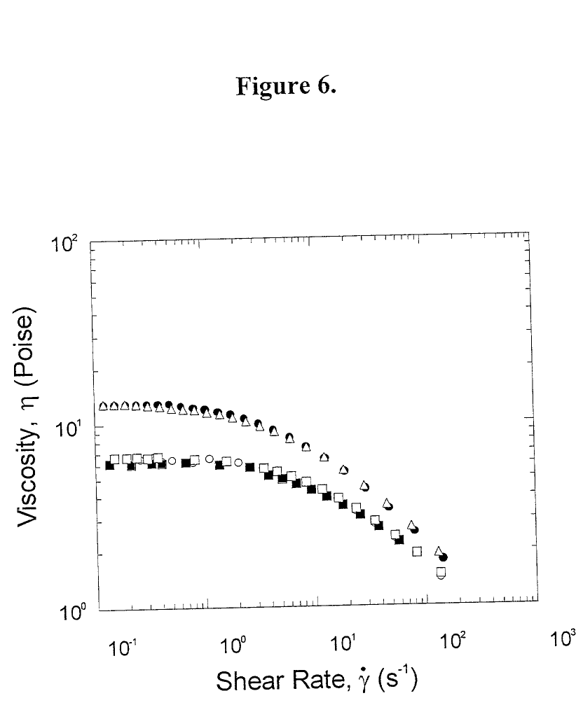

Enzyme Degradation and the Chemical Structure of TRIS: TRIS is a simple molecule with hydroxymethyl groups forming three branches of a quaternary substituted carbon with nitrogen completing the quartet. The pH-dependent inhibition suggests that the nitrogen, whose protonation state is sensitive to pH, may play a key role. To investigate the role of nitrogen in the inhibition of enzymatic degradation, experiments were conducted using available variants of TRIS with unique nitrogen substituents. The TRIS variants used were Tris (hydroxymethyl) nitromethane and N-tris (hydroxymethyl) methylglycine. The viscosity profiles of the guar solutions containing these TRIS variants and subjected to enzyme treatment for 1 hour are shown in FIG. 6. The TRIS variants displayed no inhibition with the zero shear viscosity; decreasing the same as the control with no TRIS variants or TRIS. Experiments were also conducted at various pH levels which revealed similar results (data not shown). This data showed that if access to the nitrogen was blocked using bulky substituents, the effectiveness of TRIS as an inhibitor was negated.

Since TRIS is commonly used as a buffer, it has two forms: a protonated and an unprotonated state, with the nitrogen atom either losing or gaining the proton. The relative amounts of the two forms are dependent on the pH of the solution and can be predicted by the Henderson-Hasselbach equation. Since the pKa of TRIS is 8.1 at 25° C. (Robyt and White, Biochemical Techniques. (1987) Prospect Heights, Ill.: Waveland Press, Inc. pg. 37), one possible explanation is that only the unprotonated form of TRIS inhibits the enzyme but not the protonated form. This type of inhibition behavior would be similar to other glycosyl hydrolase inhibitors based on nitrogen containing sugar analogs.

Previous work with nitrogen-containing sugar analogues have shown that a small group of glycosyl hydrolases are inhibited by the cationic (protonated) form while most are inhibited by the basic form (unprotonated) (Legler and Finken, Carbohydrate Research, 292 (1996) 103-115). An unprotonated (basic form) inhibitor was able to accept a proton from a catalytic amino acid creating a favorable electrostatic interaction between the enzyme and the inhibitor (Caron and Withers, Biochemical And Biophysical Research Communications, 163 (1989) 495-499; Legler, Pure and Applied Chemistry, 59 (1987) 1457-1464). A protonated (cationic) inhibitor forms an ion pair with negatively charged carboxylic acid residues in the active site of the enzyme.

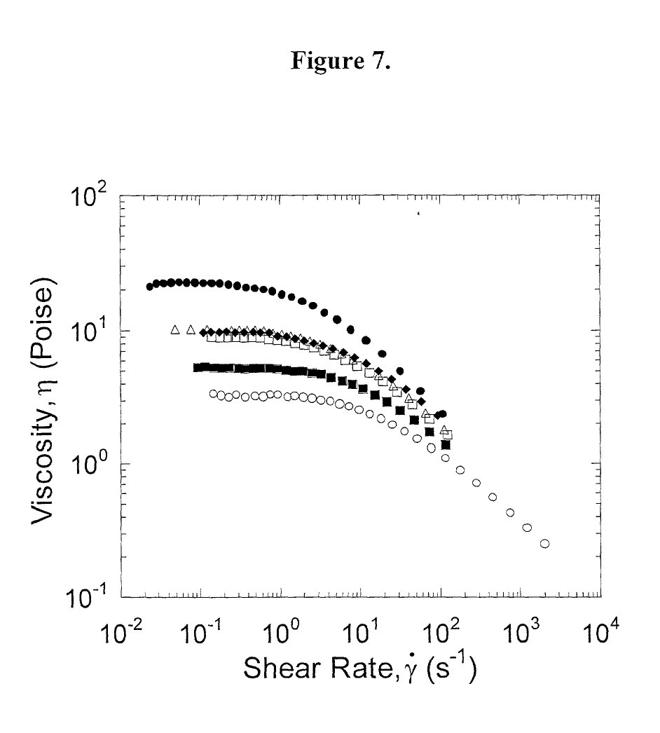

The inhibition experiments at pH 4 and 9 suggest that the basic (unprotonated) form of TRIS was inhibiting the enzyme. To verify this, the effect of TRIS on viscosity reduction was investigated at pH 7 and the results are shown in FIG. 7. At pH 7, it is expected that the enzyme would not be inhibited by TRIS since it is predominately in its cationic (protonated) form. However, it was found that at low concentrations, TRIS was a better inhibitor of the enzyme than at pH 9. This suggests that both forms of TRIS can act as inhibitors but the cationic form was a more effective inhibitor. The results in FIG. 7 may seem to be unexpected because no inhibition of enzymatic degradation was observed at pH 4, however, the effect of pH on the enzyme itself should be considered. A pH shift from 7 to 4 can change the protonation state of the amino acids in the active site of the enzyme. Although amino acid side chain pKa values are sensitive to the microenvironment of the active site, based on standard pKa values there are only two amino acids that have functional groups with pKa values between 4 and 9, histidine (pKa=6) and cysteine (pKa =8.3) (Voet and Voet, Biochemistry. 2nd ed. 1995, Somerset, N.J.: John Wiley & Sons, Inc. 254-255). Therefore, a histidine residue is a potential amino acid, which may be responsible for the unusual pH-dependent inhibition observed at pH 4 and 7.

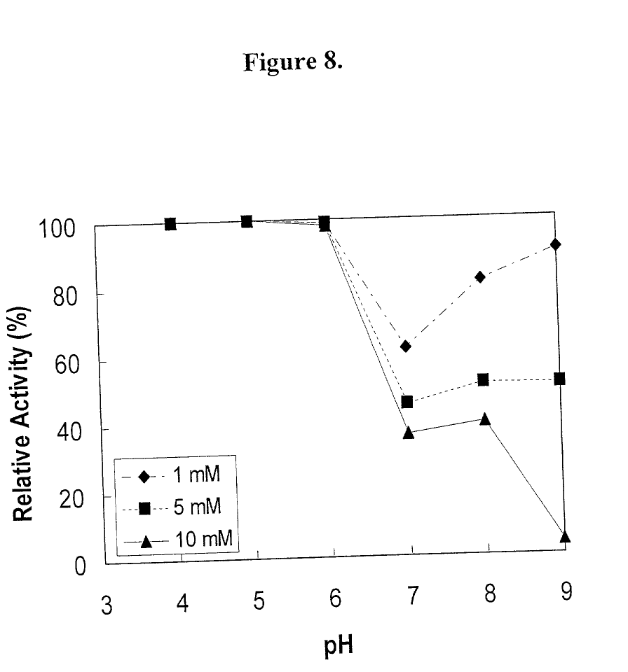

Insights into the Activity Site: The pH-dependent inhibition of β-mannanase by TRIS was quantified from pH 4 to pH 9 to further understand the pH dependency. Since the activity of the enzyme was pH dependent as well, an attempt to de-couple this effect from the effect of TRIS, by normalizing the relative activity for pH, was made. The relative activity was defined as outlined in the experimental section. FIG. 8 shows the relative activity as a function of pH for guar containing different amounts of TRIS. It was found that at pH 6 and below, no inhibition was observed. Above pH 6, adding 1 or 5 mM TRIS produced significant inhibition that had a minimum relative enzyme activity (or conversely maximum inhibition) at pH 7. This suggested that the most effective mechanism of inhibition was the formation of an ion pair between the negatively charged carboxylic amino acid and the positively charged TRIS, which was consistent with the majority of nitrogen based sugar analog inhibitors. Although both forms of TRIS could inhibit β-mannanase, if the cationic form were truly the better inhibitor then it raised the question as to why inhibition was not observed below pH 7.

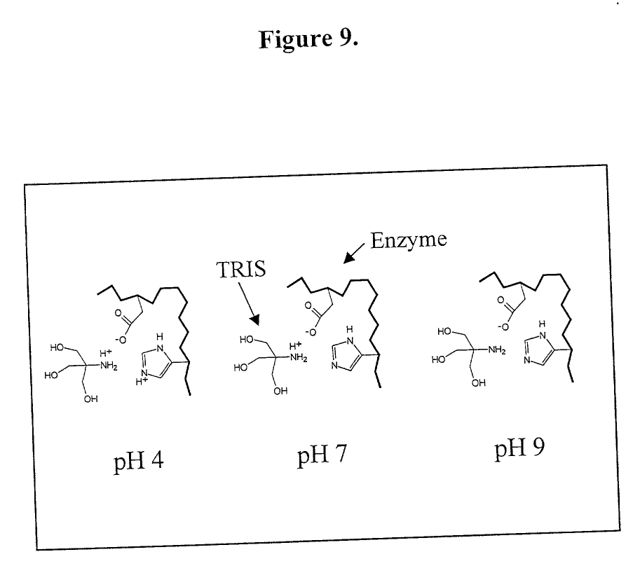

To explain this unusual pH dependent inhibition, the key amino acid residues located in the active site of the enzyme were examined. β-mannanases are categorized as glycosyl hydrolases and X-ray crystallography experiments have shown that a histidine or asparagine residue often stabilizes the position and protonation state of the catalytic amino acids for glycosyl hydrolases or plays a key role in transition state binding (Sulzenbacher et al., Biochemistry, 36 (1997) 5902-5911; Hilge et al., Structure with Folding and Design, 6 (1998) 1433-1444). In either case, mutations of this residue lead to complete loss of activity (Hilge et al., Structure with Folding and Design, 6 (1998) 1433-1444). By comparing the amino acid sequence of various β-mannanases, it has been revealed that family 26 invariably has a histidine residue followed by the acid-base catalytic amino acid (Bolam et al., Biochemistry, 35 (1996) 16195-16204) and family 5 invariably has an asparagine residue followed by the acid-base catalytic amino acid (Henrissat et al., Proc. Natl. Acad. Sci. USA, 92 (1995) 7090-7094; Hilge et al., Structure with Folding and Design, 6 (1998)1433-1444). Unfortunately, the amino acid sequence of Aspergillus niger β-mannanase has not been determined, thus preventing a priori enzyme family classification. However, based on the pH dependent inhibition, the implication is that a histidine residue precedes the acid-base catalytic amino acid due to the onset of inhibition above pH 6. When the imidazole ring of the histidine was protonated, the positive charge repels the positively charged TRIS (FIG. 9, pH 4 ). However, when the histidine residue became unprotonated (FIG. 9, pH 7 and 9), the electrostatic repulsion was not present and the TRIS was an effective inhibitor.

When the concentration of TRIS was increased to 10 mM (FIG. 8), an inflection point continued to be observed at pH 7, which was consistent with the 1 and 5 mM TRIS data. However, at pH 9 there wais a drastic reduction in enzyme activity, much greater than the inhibition at pH 7, which was not found when using 1 or 5 mM TRIS. The concentration dependence was somewhat unclear, but previous researchers have stated that Aspergillus niger β-mannanase is only stable between pH 3-8 (Ademark et al., Journal of Biotechnology, 63 (1998) 199-210; McCleary, Soluble, Dye-Labeled Polysaccharides for the Assay of Endohydrolases, in: W. A. Wood, S. T. Kellogg, (Ed.), Methods in Enzymology, Academic Press, Inc., San Diego, 1988, p. 74-87). To address this issue, the enzyme was placed in a pH 9 solution for 5 hours, then its viscosity reduction ability was compared to the previously performed 5 hour degradation. By replacing the ηo (w/Enzyme, w/TRIS) variable with no (w/Enzyme,w/pH 9 treatment) the relative activity definition found in the experimental section can be applied. After 5 hours in a pH 9 solution, the relative activity of the enzyme was 83%. Due to the reduced stability of the enzyme at high pH, the increased inhibition at 10 mM TRIS and pH 9 may be the result of a combined inhibition and destabilizing effect on the enzyme by the abundance of TRIS in solution.

### EXAMPLE 2

### Triggered Enzymatic Degradation Using Cationic Modified Guar

Experimental: A sample of cationic guar with a degree of substitution (DS) 0.14 was supplied by Rhodia Inc. (Cranbury, N.J.). The degree of substitution was defined as the average number of cationic groups substituted per sugar unit. The polymer solutions were prepared in the following manner. A mixing impeller was adjusted about 2 mm above the bottom of a 1000 ml wide mouth jar containing 150 ml deionized water. The speed of the mixing impeller was increased to 1000 rpm to form a deep vortex. Then 1 g guar powder was sprinkled slowly into the water in three minutes to produce a uniform dispersion and allowed to mix for five minutes. Another 49 ml deionized water was added to wash all residual powder into the solution. The mixing speed was then reduced to 500 rpm for an additional 60 minutes. After the speed was reduced, 100 ppm of sodium azide (FisherChemical) was added as a preservative. The solution pH was adjusted to 7.0 using HCl (EM Science). Finally, the polymer solution was transferred to a container and then placed on a low shear roller for approximately 20˜24 hours. The solution was stored in a refrigerator after being taken off the roller.

The enzyme endo-β-mannanase from aspergillus niger (Megazyme) was supplied as an ammonium sulphate suspension in 0.02% sodium azide. To prepare solutions, 0.01 ml of this suspension was diluted 1,000 times in 10 ml 0.1 M sodium acetate (EM Science)-acetic acid (Glacial, FisherChemical) buffer solution with pH adjusted to 6.

The enzymatic degradation reaction was run in a sealed jar at room temperature. The pH of the solution was measured and adjusted before the reaction. Into 200 ml of guar solution, 0.135 ml of enzyme buffer solution was injected using a microsyringe. The mixture was magnetically stirred during the reaction. After the reaction began, aliquots of the guar and enzyme mixture were taken out at various times. Each aliquot was immediately heated to 100° C. for 20 minutes to denature the enzyme and stop the reaction. The viscosity of the solution did not change after the enzyme was denatured. Experiments were also done to show that this denaturing protocol (i.e. heat treatment) did not produce a viscosity reduction for polymer solutions in the absence of enzymes. In order to compare results from different experiments, equal endo β-mannanase concentration was used as a basis. Degradation experiments were also run at different solution ionic strengths. The ionic strength was adjusted using sodium chloride (Aldrich).

Steady shear rheological tests on a strain-controlled rheometer (RFS-II, Rheometrics, Piscataway, N.J.) were used to characterize the reaction samples. A Couette geometry, with inner bob and outer cup radii of 16 mm and 16.925 mm, respectively, and a bob length of 33.3 mm, was chosen. Samples without enzyme were also tested as controls to assess the initial viscosity of the solutions. All viscosity measurements were made at 25° C.

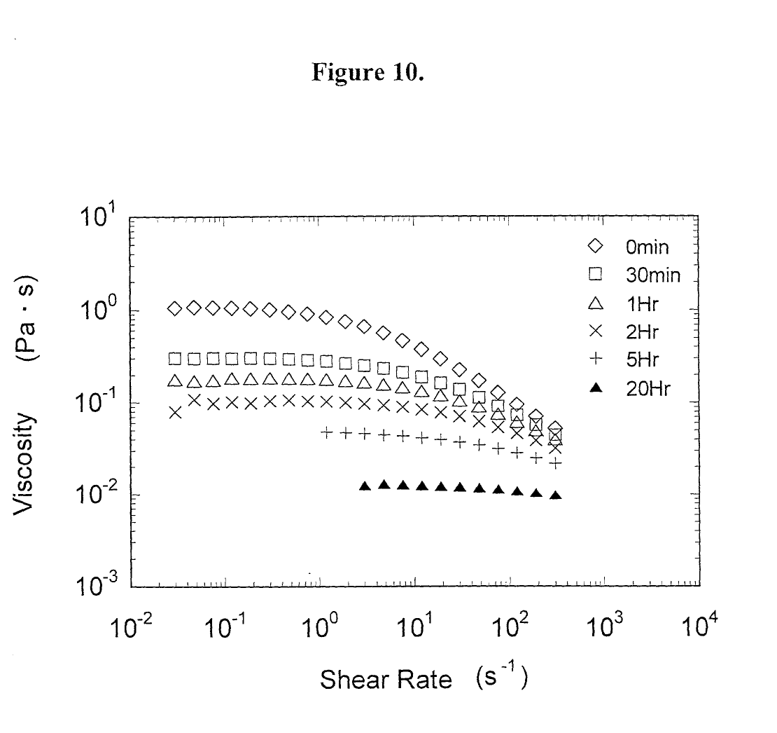

Results: FIG. 10 shows the viscosity versus shear rate plot for a 0.5 wt % guar solution upon exposure to β-mannanase enzyme with a concentration of 0.0002 units/ml guar solution. Under enzymatic hydrolysis, the solution viscosity decreased over two orders of magnitude after twenty hours. The samples displayed a Newtonian region at low shear rates and a shear-thinning region at higher shear rates. Therefore, β-mannanase is very active in degrading guar polymer chains at neutral pH.

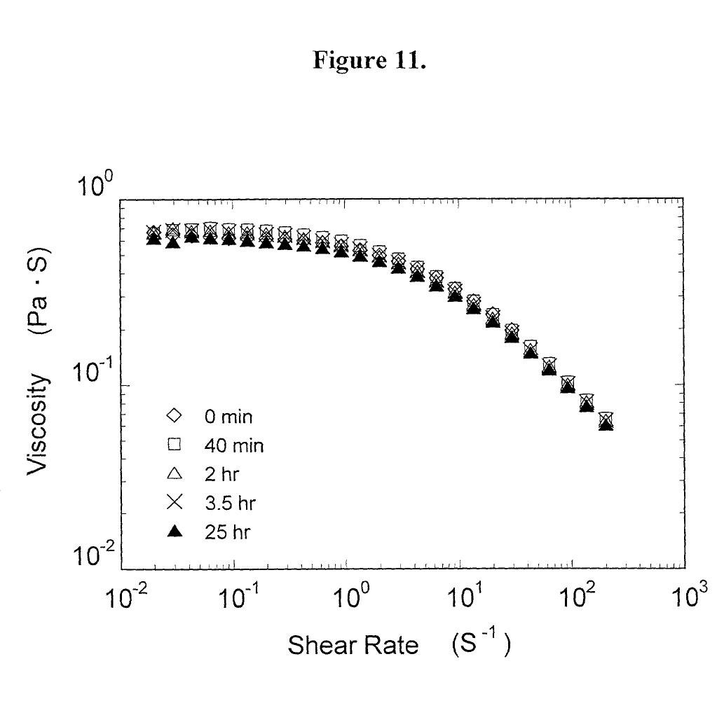

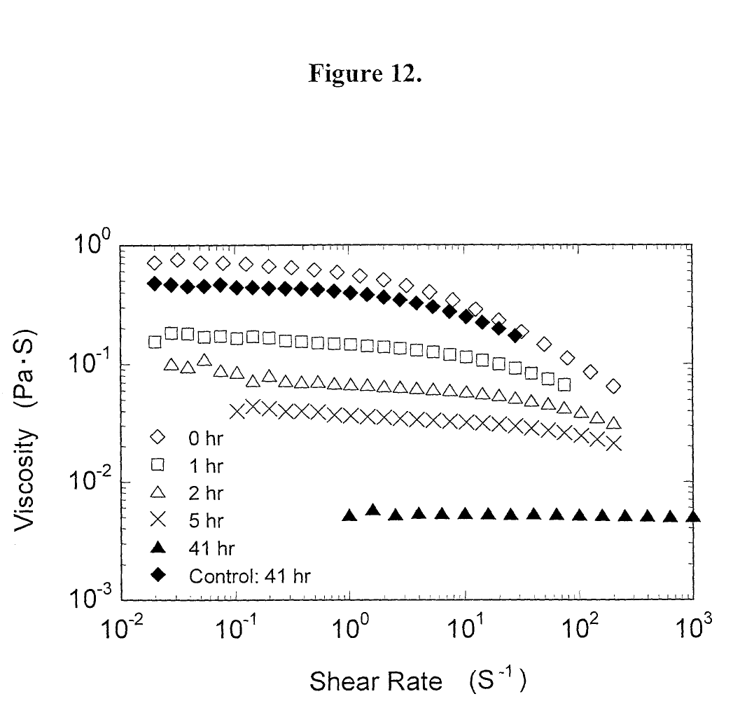

The viscosity versus shear rate plots of cationic guar solutions mixed with β-mannanase at pH 7.0 is plotted in FIG. 11. The viscosity of the solution did not change over a twenty-hour period: the enzymatic degradation was prevented. When the pH was lowered to the isoelectric point of β-mannanase (pH=3.5) (Megazyme International Ireland Ltd.), degradation began immediately as shown in FIG. 12. Since the acetal linkages between the mannose can hydrolyze at low pH, a control curve was run to see the effect of acid-catalyzed cleavage of the polymer backbone at pH 3.5 without added enzyme (FIG. 12, ♦ symbol). The viscosity of cationic guar solution decreased dramatically by two orders of magnitude over forty hours. The control solution viscosity dropped from 0.75 Pa·s to 0.45 Pa·s due to the acid-catalyzed cleavage of polymer molecules over the same period. This demonstrated a pH-activated trigger to control enzyme activity. At high pH, the negatively charged enzyme formed a complex with the cationic polymer, and this reversible immobilization prevented enzymatic action. As the isoelectric point of the β-mannanase is 3.5 (Megazyme International Ireland Ltd.), lowering the pH to 3.5 disrupted the Coulombic complex and initiated degradation.

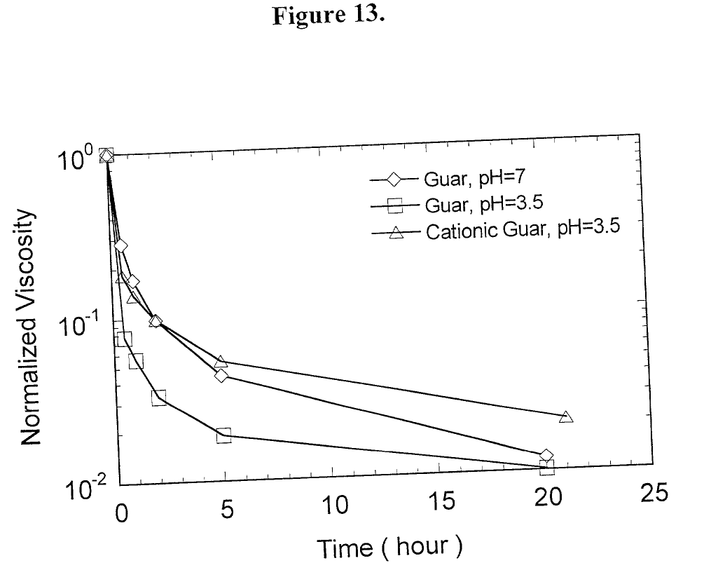

The control of degradation kinetics by pH for the β-mannanase/cationic guar pair was not due to the pH sensitivity of the enzyme activity. This is demonstrated FIG. 13, which shows that there was substantial viscosity reduction for neutral guar at both pH 7 and 3.5. The enzyme showed high activity on the substrate at pH 7. However, the degradation was completely hindered for cationic guar at neutral pH. The pH optimum for β-mannanase, with neutral guar, was around 3.0 (data not shown). The degradation rate for the enzyme, with cationic guar at pH 3.5, was about at half the rate as with neutral guar. This may be due to residual Coulombic interactions or the steric inhibition of enzyme binding by the cationic graft site.

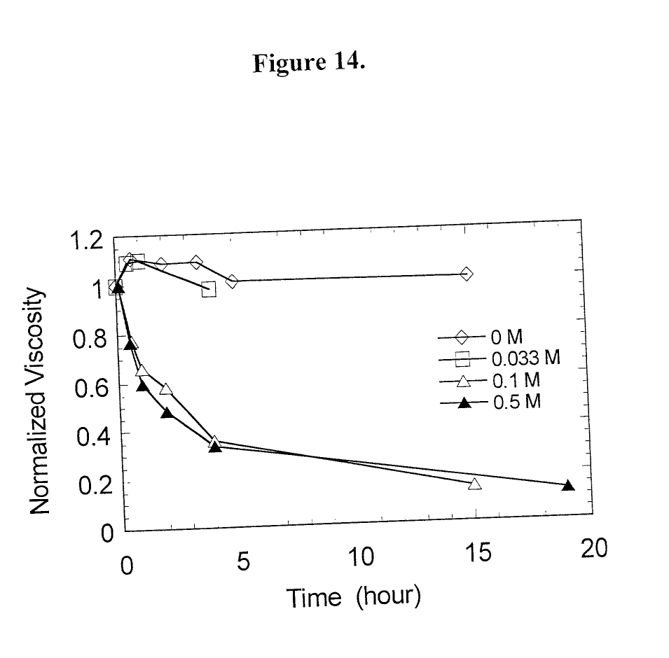

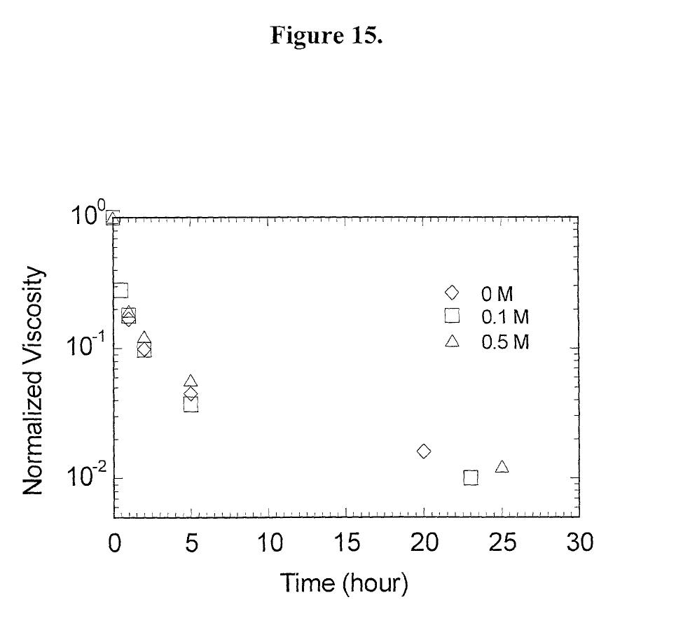

Charge complex triggering was further evaluated at a constant pH of 7.0. FIG. 14 shows the effectiveness of the β-mannanase in degrading cationic guar solutions at different ionic strengths but a constant pH of 7.0. At low ionic strength, the enzymatic degradation was stopped. When the ionic strength was increased to 0.1 M, the solution viscosity decreased substantially. As the solution ionic strength increased, the electrostatic attraction between the enzyme molecule and the polymer chain was screened and the complex was disrupted. A control experiment was run on native guar to show that salt concentration has little effect on enzyme activity (FIG. 15).

Therefore, the enzyme activity is inhibited by adding a HMW polymer agent. The HMW agent and the enzyme must be of opposite charge in the “arrested” state. The enzyme can be either negatively or positively charged, depending on the solution pH. The degradation can be initiated either by shifting pH to alter the charge on either the polymer or enzyme, or by adding salts (of any anion or cation type) to free complexed enzyme to initiate degradation. Examples of oppositely charged HMW agents would include sulfated, phosphated, carboxylated or amine, quaternary amine or sulfonium ion containing synthetic or natural polymers.

The foregoing examples are illustrative of the present invention, and are not to be construed as limiting thereof. The invention is described by the following claims, with equivalents of the claims to be included therein.

That which is claimed is:

1. A method of fracturing a subterranean formation which surrounds a well bore, comprising the steps of:
(a) providing a fracturing fluid comprising (i) an aqueous liquid; (ii) a polysaccharide soluble or dispersible in said aqueous liquid in an amount sufficient to increase the viscosity of said aqueous liquid; (iii) an enzyme breaker which degrades said polysaccharide; and (iv) a compound according to Formula I in an amount sufficient to reduce the polysaccharide-degrading activity of said enzyme breaker; 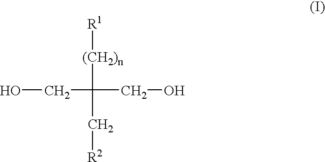
wherein:
R¹ is selected from the group consisting of: —F, —NR³R⁴ wherein R³ and R⁴ are each independently selected from the group consisting of H, loweralkyl, 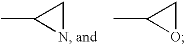
R² is selected from the group consisting of —H and —OH; and
n is 0 to 3;
then;
(b) injecting said fracturing fluid into said well bore at a pressure sufficient to form fractures in the subterranean formation which surrounds said well bore; then
(c) reducing the pH of said fracturing fluid by an amount sufficient to increase the polysaccharide-degrading activity of said enzyme; and then
(d) releasing the pressure from said fracturing fluid.

2. A method according to claim 1, wherein said enzyme is a β-mannanase.

3. A method according to claim 1, wherein said enzyme is a β-glucosidase.

4. A method according to claim 1, wherein R¹ is —NH₂.

5. A method according to claim 1, wherein R² is —OH.

6. A method according to claim 1, wherein n is 1.

7. A method according to claim 1, wherein said compound of Formula 1 is 2-amino-2-hydroxymethyl-1,3-propanediol.

8. A method according to claim 1, wherein said polysaccharide comprises guar gum or derivatives thereof.

9. A method according to claim 1, wherein said fracturing fluid further comprises proppant particles.

10. A method according to claim 1, wherein said fracturing fluid further comprises a crosslinking agent for crosslinking said polysaccharide.

11. A fracturing fluid useful for fracturing a subterranean formation which surrounds a well bore, comprising:
(i) an aqueous liquid;
(ii) a polysaccharide soluble or dispersible in said aqueous liquid in an amount sufficient to increase the viscosity of said aqueous liquid;
(iii) an enzyme breaker which degrades said polysaccharide; and
(iv) a compound according to Formula I in an amount sufficient to reduce the polysaccharide-degrading activity of said enzyme breaker; 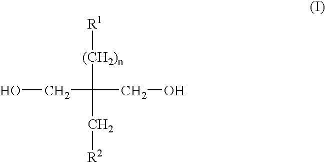
wherein:
R¹ is selected from the group consisting of: —F, —NR³R⁴ wherein R³ and R⁴ are each independently selected from the group consisting of H, loweralkyl, 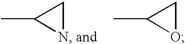
R² is selected from the group consisting of —H and —OH; and
n is 0 to 3.

12. A fracturing fluid according to claim 11, wherein said enzyme is a β-mannanase.

13. A fracturing fluid according to claim 11, wherein said enzyme is a β-glucosidase.

14. A fracturing fluid according to claim 11, wherein R¹ is —NH₂.

15. A fracturing fluid according to claim 11, wherein R² is —OH.

16. A fracturing fluid according to claim 11, wherein n is 1.

17. A fracturing fluid according to claim 11, wherein said compound of Formula 1 is 2-amino-2-hydroxymethyl-1,3-propanediol.

18. A fracturing fluid according to claim 11, wherein said polysaccharide comprises guar gum or derivatives thereof.

19. A fracturing fluid according to claim 11, wherein said fracturing fluid further comprises proppant particles.

20. A fracturing fluid to claim 11, wherein said fracturing fluid further comprises a crosslinking agent for crosslinking said polysaccharide.

21. A method of fracturing a subterranean formation which surrounds a well bore, comprising the steps of:
(a) providing a fracturing fluid comprising (i) an aqueous liquid; (ii) a polysaccharide soluble or dispersible in said aqueous liquid in an amount sufficient to increase the viscosity of said aqueous liquid; (iii) an enzyme breaker which degrades said polysaccharide, and (iv) a polymeric additive which has a positive charge to decrease the activity of said enzyme breaker under conditions where the enzyme has a negative charge, subject to the proviso that said polymeric additive and said polysaccharide may be different or the same;
(b) injecting said fracturing fluid into said well bore at a pressure sufficient to form fractures in the subterranean formation which surrounds said well bore; then
(c) reducing the pH of said fracturing fluid by an amount sufficient to increase the polysaccharide-degrading activity of said enzyme; and then
(d) releasing the pressure from said fracturing fluid.

22. A method according to claim 21, wherein said enzyme is a β-mannanase.

23. A method according to claim 21, wherein said enzyme is a β-glucosidase.

24. A method according to claim 21, said polymeric additive carrying cationic moities, said cationic moieties selected from the group consisting of amines, quaternary amines and sulfonium ions.

25. A method according to claim 21, wherein said polysaccharide comprises guar gum or derivatives thereof.

26. A method according to claim 21, wherein said fracturing fluid further comprises proppant particles.

27. A method according to claim 21, wherein said fracturing fluid further comprises a crosslinking agent for crosslinking said polysaccharide.

28. A method of fracturing a subterranean formation which surrounds a well bore, comprising the steps of:
(a) providing a fracturing fluid comprising (i) an aqueous liquid; (ii) a polysaccharide soluble or dispersible in said aqueous liquid in an amount sufficient to increase the viscosity of said aqueous liquid; (iii) an enzyme breaker which degrades said polysaccharide, and (iv) a polymeric additive which has a negative charge to decrease the activity of said enzyme breaker under conditions where the enzyme has a positive charge, subject to the proviso that said polymeric additive and said polysaccharide may be different or the same;
(b) injecting said fracturing fluid into said well bore at a pressure sufficient to form fractures in the subterranean formation which surrounds said well bore; then
(c) reducing the pH of said fracturing fluid by an amount sufficient to increase the polysaccharide-degrading activity of said enzyme; and then
(d) releasing the pressure from said fracturing fluid.

29. A method according to claim 28, wherein said enzyme is a β-mannanase.

30. A method according to claim 28, wherein said enzyme is a β-glucosidase.

31. A method according to claim 28, said polymeric additive carrying anionic moities, wherein said anionic moieties are selected from the group consisting of carboxylic, sulfate, sulfonate, phosphate and phosphonate ions.

32. A method according to claim 28, wherein said polysaccharide comprises guar gum or derivatives thereof.

33. A method according to claim 28, wherein said fracturing fluid further comprises proppant particles.

34. A method according to claim 28, wherein said fracturing fluid further comprises a crosslinking agent for crosslinking said polysaccharide.

35. A method of fracturing a subterranean formation which surrounds a well bore, comprising the steps of:
(a) providing a fracturing fluid comprising (i) an aqueous liquid; (ii) a polysaccharide soluble or dispersible in said aqueous liquid in an amount sufficient to increase the viscosity of said aqueous liquid; (iii) an enzyme breaker which degrades said polysaccharide, and (iv) a polymeric additive which has a positive charge to decrease the activity of said enzyme breaker under conditions where the enzyme has a negative charge, subject to the proviso that said polymeric additive and said polysaccharide may be different or the same;
(b) injecting said fracturing fluid into said well bore at a pressure sufficient to form fractures in the subterranean formation which surrounds said well bore; then
(c) increasing the ionic strength of said fracturing fluid by an amount sufficient to increase the polysaccharide-degrading activity of said enzyme; and then
(d) releasing the pressure from said fracturing fluid.

36. A method according to claim 35, wherein said enzyme is a β-mannanase.

37. A method according to claim 35, wherein said enzyme is a β-glucosidase.

38. A method according to claim 35, said polymeric additive carrying cationic moities, said cationic moieties selected from the group consisting of amines, quaternary amines and sulfonium ions.

39. A method according to claim 35, wherein said polysaccharide comprises guar gum or derivatives thereof.

40. A method according to claim 35, wherein said fracturing fluid further comprises proppant particles.

41. A method according to claim 35, wherein said fracturing fluid further comprises a crosslinking agent for crosslinking said polysaccharide.

42. A method of fracturing a subterranean formation which surrounds a well bore, comprising the steps of:
(a) providing a fracturing fluid comprising (i) an aqueous liquid; (ii) a polysaccharide soluble or dispersible in said aqueous liquid in an amount sufficient to increase the viscosity of said aqueous liquid; (iii) an enzyme breaker which degrades said polysaccharide, and (iv) a polymeric additive which has a negative charge to decrease the activity of said enzyme breaker under conditions where the enzyme has a positive charge, subject to the proviso that said polymeric additive and said polysaccharide may be different or the same;
(b) injecting said fracturing fluid into said well bore at a pressure sufficient to form fractures in the subterranean formation which surrounds said well bore; then
(c) increasing the ionic strength of said fracturing fluid by an amount sufficient to increase the polysaccharide-degrading activity of said enzyme; and then
(d) releasing the pressure from said fracturing fluid.

43. A method according to claim 42, wherein said enzyme is a β-mannanase.

44. A method according to claim 42, wherein said enzyme is a β-glucosidase.

45. A method according to claim 42, said polymeric additive carrying anionic moities, wherein said anionic moieties are selected from the group consisting of carboxylic, sulfate, sulfonate, phosphate and phosphonate ions.

46. A method according to claim 42, wherein said polysaccharide comprises guar gum or derivatives thereof.

47. A method according to claim 42, wherein said fracturing fluid further comprises proppant particles.

48. A method according to claim 42, wherein said fracturing fluid further comprises a crosslinking agent for crosslinking said polysaccharide.

49. A fracturing fluid useful for fracturing a subterranean formation which surrounds a well bore, comprising:
(i) an aqueous liquid;
(ii) a polysaccharide soluble or dispersible in said aqueous liquid in an amount sufficient to increase the viscosity of said aqueous liquid;
(iii) an enzyme breaker which degrades said polysaccharide;
(iv) a polymeric additive which has an opposite charge to that of the enzyme to decrease the activity of said enzyme breaker,
subject to the proviso that said polymeric additive and said polysaccharide may be different or the same.

50. A fracturing fluid according to claim 28, wherein said enzyme is a β-mannanase.

51. A fracturing fluid according to claim 28, wherein said enzyme is a β-glucosidase.

52. A fracturing fluid according to claim 28, said polymeric additive carrying an ionic group, wherein said ionic group is selected from the group consisting of sulfate, sulfonate, phosphate, phosphonate, carboxylate, amine, quaternary amine or sulfonium ions.

53. A fracturing fluid according to claim 28, wherein said polysaccharide comprises guar gum or derivatives thereof.

54. A fracturing fluid according to claim 28, wherein said fracturing fluid further comprises proppant particles.

55. A fracturing fluid to claim 28, wherein said fracturing fluid further comprises a crosslinking agent for crosslinking said polysaccharide.
